(19) United States
(12) Patent Application Publication
(10) Pub. No.: US 2003/0098028 A1
(43) Pub. Date: May 29, 2003

(54) Method for providing spinal postural biofeedback using taping

(76) Inventor: Charles Bronder, Lodi, NJ (US)

Correspondence Address: Jean-Marc Zimmerman, 226 St. Paul Street, Westfield, NJ, 07090, US

(21) Appl. No.: 09/997,881

(22) Filed: Nov. 29, 2001

(51) Int. Cl. A61B 19/00

(52) U.S. Cl. 128/897

(57) ABSTRACT

A method for providing spinal biofeedback to a patient so that the patient knows when they have moved their spine in a manner that does not comply with a clinician's instructions, which noncompliant movement may aggravate an existing injury or cause further injury. The patient's spine is taped so that when the patient moves in a noncompliant manner the skin is stimulated as a result of the tape pulling on the skin thereby providing the biofeedback to the patient. By examining the condition of the tape on a subsequent visit to observe if there are any deformations, the clinician is able to determine if the patient has moved in a noncompliant manner. The taping may be applied by a clinician after the patient's spine has been decompressed, manipulated, mobilized, and/or massaged. Taping can be used in conjunction with therapeutic devices, such as braces, and/or in conjunction with other therapies, such as therapeutic activities and/or exercises.

### FIELD OF THE INVENTION

The present invention pertains to the field of biofeedback techniques, and more particularly to a method for providing 24 hour direct spinal postural biofeedback to the patient.

### BACKGROUND OF THE INVENTION

Physical medicine treatments are widely used by clinicians, e.g., chiropractors, physical therapists, athletic trainers, nurses, osteopathic and allopathic physicians, in order to treat pain and/or dysfunction commonly associated with musculoskeletal disorders, conditions and/or injuries, such as back and/or neck disorders, conditions, and/or injuries. Such treatments may restrict the patient from participating in and/or performing certain activities and/or joint movements achieved through patient instructions and/or bracing or taping techniques. Others may be directed at attempting to relieve pain through the restoration of normal joint function/movement.

However, such conventional techniques suffer from several drawbacks. First, once a patient leaves a clinician's office, no mechanism exists for informing the patient when they move in a manner that does not comply with the clinician's instructions and which can aggravate an existing injury or otherwise injure the patient. Second, once the patient leaves the clinician's office, the clinician is unable to determine whether the patient has moved in a non-compliant manner. It is therefore an object of this present invention to provide a method which to: A) maximize the patient's desire/potential to co-operate, comply, and participate in a course of treatment plan; and B) maximize the clinician's ability to accurately monitor patient compliance, co-operation and participation in the course of treatment.

### SUMMARY

A method for providing spinal biofeedback to a patient so that they know when they have moved their spine in a manner that does not comply with a clinician's instructions and may subsequently aggravate the patient's condition, disorder, and/or injury. The patient's spine is taped so that when the patient moves in a non-compliant manner the skin is stimulated as a result of the tape pulling on the skin, thereby providing biofeedback so that the patient knows they moved inappropriately. By examining the condition of the tape to observe any deformations on a subsequent patient visit, the clinician is able to determine if the patient has moved in a non-compliant manner. The taping may be applied by a clinician after the patient's spine has been decompressed, manipulated, mobilized, and/or massaged. The taping can be performed as often as deemed necessary by a clinician. Taping can be used in conjunction with devices, such as braces, and/or in conjunction with other therapies, including therapeutic exercises.

### BRIEF DESCRIPTION OF THE DRAWINGS

FIG. 1 shows a flowchart depicting the steps by which an exemplary embodiment of the present invention operates.

### DETAILED DESCRIPTION OF THE INVENTION

The present invention discloses a method for providing spinal postural biofeedback to a patient by applying tape to the patient's spine. Such spinal taping is performed as part of a course of treatment prescribed by a clinician to enable the patient to avoid non-compliant movements of the spine and to enable the clinician to monitor the patient's progress so that the patient can be successfully treated. Taping helps minimize the potential for re-injury by restricting spinal movement that occurs during common daily activities, such as brushing ones teeth, or getting dressed etc.

Spinal taping serves three essential functions. First, such taping provides the patient with 24 hour direct spinal postural biofeedback which is essential in attempting to prevent further re-injury and/or exacerbation of the condition/disorder. Taping achieves this end while minimizing any loss of muscular stabilization tone commonly associated with prolonged use of a brace. This postural biofeedback is provided whether the patient is awake or sleeping.

A patient's postural/spinal kinesthetic biofeedback control is essential in order to prevent spinal re-injuries and/or exacerbation of such injuries, conditions and/or disorders that may occur when the patient moves in a manner that does not comply with the clinician's instructions. Such compromised spinal positions are often attained when a patient moves while asleep. The taped patient, whether asleep or awake, will experience biofeedback in the form of skin stimulation resulting from the tape pulling the skin to which it is attached, which shall alert, e.g., wake the patient to modify their sleeping posture/position so as to reduce and/or eliminate any associated potential for re-injury/exacerbation commonly attributed to sleeping in an un-intentional non-compliant spinal position.

Second, spinal taping provides a clinician with an indicator by which they can monitor the degree to which a patient has complied with the clinician's instructions regarding limiting their movement or maintaining a particular spinal posture. Patients that ignore the clinician's instructions are easy to separate from compliant patients due to the wrinkling and/or detachment of the tape worn by the non-compliant patient. Being able to determine whether and the extent to which a patient complies with the clinician's instructions is essential in order to maximize the therapeutic benefit of a therapeutic procedure.

Third, spinal taping provides the means by which a clinician can adjust and/or modify the patient's posture on subsequent visits to a more anatomically appropriate position. This can not be reasonably achieved through the use of any conventional brace without sacrificing muscular tone integrity. Although taping can be performed as often, e.g., daily, weekly, etc., as deemed necessary by a clinician to treat a patient, it is most effective if performed on a daily basis. Taping enables the clinician to modify treatment of the patient proportionate to the patient's progress so as to restore a normal lumbar lordotic curvature or disc angle. By gently manipulating, mobilizing, massaging, or through any therapeutic modality or activity the clinician can modify the patient's spinal posture before taping the patient on each subsequent visit, thus attempting to maintain said therapeutic gain or postural correction, the clinician can gently retrain the movements of the patient's spine so that they use proper physical bio-mechanics when moving, especially when sitting and when rising from a sitting position, thereby speeding up the recovery process.

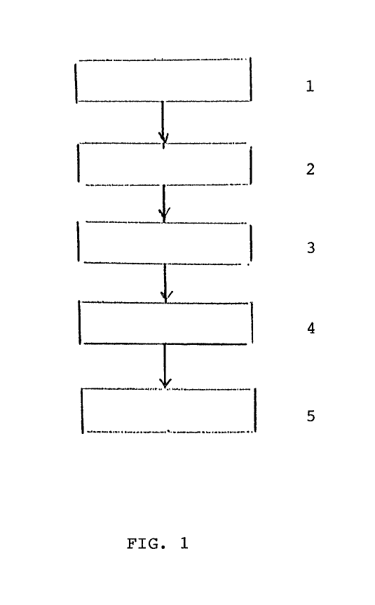

FIG. 1 shows a flowchart depicting the steps by which taping a patient's back is used to provide spinal biofeedback according to an exemplary embodiment of the present invention. At step 1, the clinician positions the patient so that the tape can be applied. This can be done by having a patient position or arch their back/spine, bend over, or move and/or orient their body and spine in any manner desired by the clinician. At step 2, the clinician applies an under-tape or pre-tape in order to protect the patient's skin. This under-tape is preferably an hypo-allergic type of tape. At step 3, the clinician applies an over-tape on top of the under-tape. The over-tape is preferably an extremely rigid type of therapeutic tape.

At step 4, the clinician observes the condition of the tape at a subsequent visit to determine if the patient has moved in a non-compliant manner. At step 5, the clinician re-tapes the patient. This is preferably done through the application of a completely new dressing of under-tape and over-tape, although it may be achieved by taping over or on top of the existing taping. When the patient is re-taped, the clinician can have the patient position their spine in an orientation that differs from that assumed during the previous taping.

The under-tape and over-tape are applied as desired by the clinician. Specifically, the tapes can be cut to any desired lengths and widths. The clinician can apply as many strips and as many layers of each tape as desired and can apply the tape in any desired orientation with respect to the vertical position of the spine so that proper biofeedback is provided to the patient and the doctor is able to observe any deformations of the applied tape that would indicate non-compliant and thus harmful movements by the patient.

Numerous modifications to and alternative embodiments of the present invention will be apparent to those skilled in the art in view of the foregoing description. Accordingly, this description is to be construed as illustrative only and is for the purpose of teaching those skilled in the art the best mode of carrying out the invention. Details of the embodiment may be varied without departing from the spirit of the invention, and the exclusive use of all modifications which come within the scope of the appended claims is reserved.

What is claimed is:

1. A method for providing spinal biofeedback to a patient, comprising the steps of:
physically positioning a patient so that their spine is in an orientation desired by a clinician;
applying an undertape to the spine; and
applying an overtape to the spine, wherein the combination of the undertape and overtape stimulates the skin of the patient when the patient moves in a manner that is noncompliant with instructions issued by the clinician thereby providing biofeedback to the patient whenever they move in a noncompliant manner, and wherein the tape deforms as a result of said noncompliant movement thereby enabling the clinician to determine that the patient has moved in a noncompliant manner.

2. The method according to claim 1, further comprising the step of periodically removing the undertape and overtape and applying a new undertape and a new overtape.

3. The method according to claim 2, further comprising the step of modifying the spinal posture of the patient before the new undertape and the new overtape are applied.

4. The method according to claim 1, wherein the stimulation is caused by the tape pulling on the skin of the patient when the patient moves in a noncompliant manner.

5. The method according to claim 1, wherein the undertape is applied to skin over the spine and the overtape is applied on top of the undertape.

6. The method according to claim 1, wherein the undertape is an hypo-allergic tape that does not tear the skin of the patient.

7. The method according to claim 1, wherein the overtape is an extremely rigid therapeutic tape.

8. The method according to claim 1, wherein the undertape and the overtape are applied parallel to another.

9. The method according to claim 1, wherein the undertape and the overtape are applied perpendicular to another.

10. The method according to claim 1, wherein a plurality of strips of undertape are applied to the patient.

11. The method according to claim 1, wherein a plurality of strips of overtape are applied to the patient.

12. The method according to claim 1, wherein the undertape and the overtape are used in conjunction with a brace.

13. The method according to claim 1, wherein the undertape and the overtape are used in conjunction with a decompression of the spine.

14. The method according to claim 1, wherein the undertape and the overtape are used in conjunction with a manipulation, mobilization, or massage of the spine.

15. The method according to claim 1, wherein the undertape and the overtape are used in conjunction with therapeutic activities and/or exercises of the spine.

16. A method for providing spinal biofeedback to a patient, comprising the steps of:
instructing a patient to assume a particular spinal posture; and
applying a strip of tape to the spine, wherein the tape stimulates the skin of the patient whenever the patient moves in a manner that is noncompliant with instructions issued by a clinician, thereby providing biofeedback to the patient when they move in a noncompliant manner.

17. The method according to claim 16, wherein the tape deforms as a result of noncompliant movement by the patient, thereby enabling the clinician to determine that the patient has moved in a noncompliant manner.

18. The method according to claim 16, further comprising the step of periodically removing the tape and applying a new tape.

19. The method according to claim 18, further comprising the step of modifying the spinal posture of the patient before the new tape is applied.

20. The method according to claim 16, wherein the stimulation is caused by the tape pulling on the skin of the patient when the patient moves in a noncompliant manner.
